(19) United States
(12) Patent Application Publication
(10) Pub. No.: US 2015/0251022 A1
(43) Pub. Date: Sep. 10, 2015

(54) RADIOTHERAPEUTIC APPARATUS

(71) Applicant: Haifeng Liu, Xi'an (CN)

(72) Inventor: Haifeng Liu, Xi'an (CN)

(21) Appl. No.: 14/430,855

(22) PCT Filed: Apr. 8, 2013
(86) PCT No.: PCT/CN2013/073854

§ 371 Date: Mar. 24, 2015

(51) Int. Cl. A61N 5/10

(52) U.S. Cl. CPC A61N 5/1081

(57) ABSTRACT

A radiotherapeutic apparatus comprises a base, a rotatable gantry, two treatment heads, and a treatment table. Wherein the rotatable gantry is disposed on the base, the treatment heads are disposed on the rotatable gantry. The radiotherapeutic apparatus further comprises driving units, gears, and arc-shaped racks. The driving units are disposed on the rotatable gantry. The gears are disposed on the rotatable gantry and driven by the first driving units. The arc-shaped racks are disposed on the treatment heads and meshes with the gears. When the gears are driven under driving of the driving units, each treatment head which is locked to the arc-shaped racks separately slide on the arc-shaped racks, along an axial direction of the rotatable gantry.

### TECHNICAL FIELD

The present invention relates to the field of medical devices, and in particular, to a radiotherapeutic apparatus.

### BACKGROUND

A conventional radiotherapeutic apparatus consists of a vertical frame, a rotary frame, a treatment head, a collimator and a treatment table. The vertical frame is coupled to the rotary frame by bearings, and the treatment head and the collimator are disposed on the rotary frame. A prefabricated lead block disposed in the collimator is shape-adaptive. A lead block needs to be fabricated at each time a treatment plan is formulated for a radiation field. A patient requires 3 to 5 radiation fields, and accordingly 3 to 5 lead blocks are needed. In addition, in order to maintain a uniform treatment dose at the field center, a ray flattening block needs to be added. In this way, more accessories are used and operations are complicated accordingly. Because the treatment head is rotatable only on a revolution surface, the rays act on a limited skin area. Consequently, exposure of the limited skin area to high ray concentration may cause damages to skin.

### SUMMARY

The present invention is directed to providing a radiotherapeutic apparatus, wherein two treatment heads separately swing along an axial direction of a rotatable gantry, thereby improving the focal skin dose ratio.

A radiotherapeutic apparatus is provided, comprising a base, a rotatable gantry, two treatment heads and a treatment table, the rotatable gantry being disposed on the base, and the treatment head being disposed on the rotatable gantry, wherein the radiotherapeutic apparatus further comprises: two groups of driving units, disposed on the rotatable gantry and coupled to the two treatment heads respectively; two groups of gears, disposed on the rotatable gantry and coupled to the driving units respectively, the driving units driving the gears to move; and two groups of arc-shaped racks, disposed on the treatment heads and meshing with the two groups of gears respectively; wherein when driving the gears, under driving of the gears, the driving units separately swing, together with the two treatment heads secured by the arc-shaped racks, along an axial direction of the rotatable gantry.

Preferably, the radiotherapeutic apparatus further comprises: arc-shaped guide rails, disposed on the rotatable gantry, a center of circle of the arc-shaped guide rail coinciding with a center of circle of the arc-shaped rack; and at least one sliding block, disposed on the treatment heads, the sliding block being slidable on the arc-shaped guide rail.

Preferably, an angle of the swinging along the axial direction of the rotatable gantry is in a range from 0 to 40 degrees.

The radiotherapeutic apparatus according to the present invention improves the focal skin dose ratio by enabling two treatment heads 3, 3′ to swing along the axial direction of the rotatable gantry and to move on two spatial revolution surfaces. In addition, a combination of the two treatment heads 3, 3′ may also improve the dose ratio at a tumor target.

### BRIEF DESCRIPTION OF THE DRAWINGS

FIG. 1 is a schematic structural view of a radiotherapeutic apparatus according to an embodiment of the present invention;

FIG. 2 is a left partial view of the radiotherapeutic apparatus according to an embodiment of the present invention;

FIG. 3 is a partially enlarged view of the radiotherapeutic apparatus according to an embodiment of the present invention; and

FIG. 4 is a sectional view of the radiotherapeutic apparatus according to an embodiment of the present invention.

### DETAILED DESCRIPTION

To make the objectives, technical solutions, and advantages of the present invention clearer, the present invention is further described in detail in combination with specific embodiments and attached drawings. It should be understood that the embodiments described here are only exemplary ones for illustrating the present invention, and are not intended to limit the present invention.

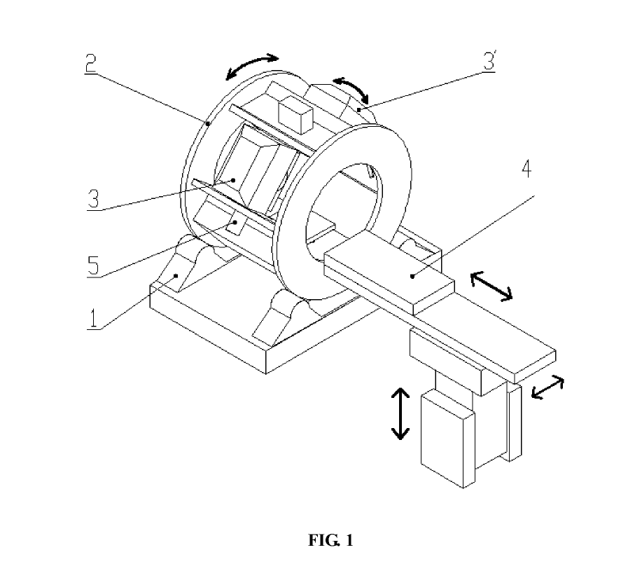

An embodiment of the present invention provides a radiotherapeutic apparatus. As illustrated in FIG. 1, the radiotherapeutic apparatus comprises a base 1, a rotatable gantry 2, two treatment heads 3, 3′ and a treatment table 4. The treatment table 4 is disposed on the base 1, and the rotatable gantry 2 is also disposed on the base 1 and coupled to the treatment table 4. The treatment heads 3, 3′ are disposed on left and right sides of a radial cross section of the rotatable gantry 2. The treatment heads 3, 3′ are provided with a radioactive source, wherein the radioactive source may be a cobalt source or other radioactive elements.

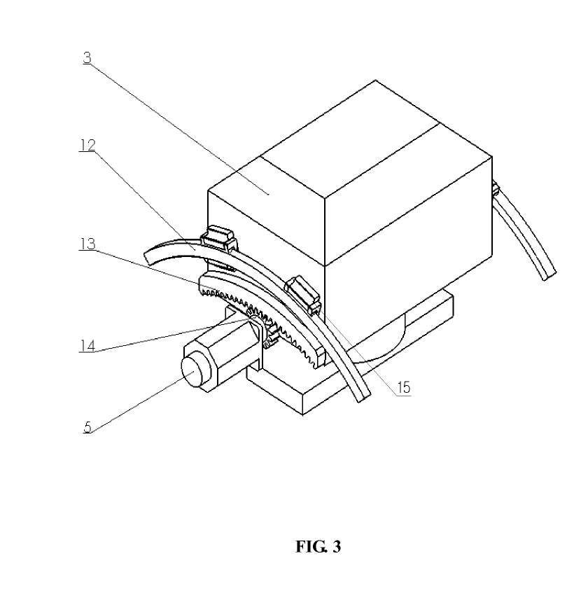

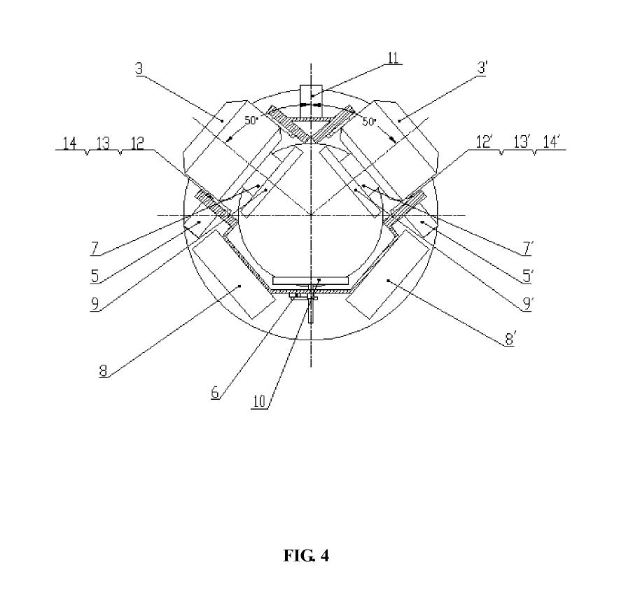

Referring to FIGS. 3 and 4, it should be noted that the radiotherapeutic apparatus further comprises two sets of driving units 5, 5′, wherein the driving units 5, 5′ are disposed on the rotatable gantry 2 and coupled to the treatment heads 3, 3′ respectively. In addition, the radiotherapeutic apparatus further comprises gears 14, 14′, arc-shaped racks 13, 13′, and arc-shaped guide rails 12, 12′, and sliding block 15, 15′. The gears 14, 14′ are disposed on the rotatable gantry 2 and coupled to the driving units 5, 5′ respectively. The arc-shaped racks 13, 13′ are disposed on the treatment heads 3, 3′. The arc-shaped guide rails 12, 12′ are disposed on the rotatable gantry 2. And the sliding blocks 15, 15′ are disposed on the rotatable gantry and slidable on the arc-shaped guide rails 12, 12′.

It should be noted that the gears 14, 14′ are engaged with the arc-shaped racks 13, 13′ respectively. In addition, centers of circles of the arc-shaped guide rails 12, 12′ coincide with the centers of circles of the arc-shaped racks 13, 13′, respectively. The arc-shaped guide rails 12, 12′ and the sliding blocks 15, 15′ are mainly used to support the treatment heads 3, 3′.

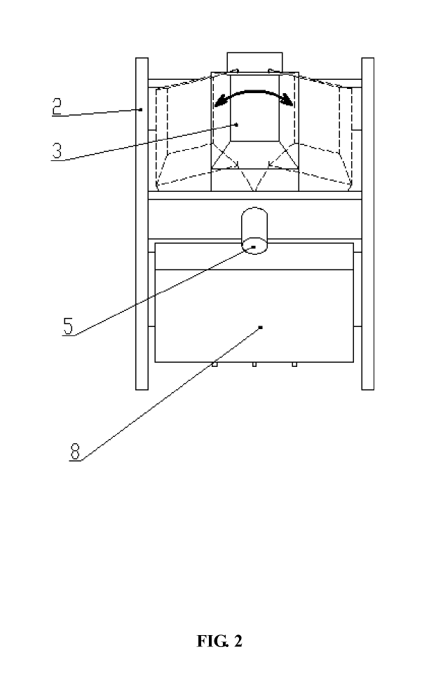

In practice, the driving units 5, 5′ respectively drive the gears 14, 14′ to rotate. As such, the arc-shaped racks 13, 13′ respectively engaged with the gears 14, 14′ are further driven to rotate, such that the treatment heads 3, 3′ securely coupled to the arc-shaped gears 13, 13′ slide/swing along the arc-shaped guide rails 12, 12′ respectively. Particularly, as illustrated in FIG. 2 which is a left view of FIG. 1, the treatment heads 3, 3′ slide/swing along an axial direction of the rotatable gantry 2, with a swinging amplitude of 0 to 40 degrees.

In addition, as illustrated in FIG. 2, the radiotherapeutic apparatus further comprises counterweight blocks 8′, 8′. The counterweight blocks 8′, 8′ are disposed on the rotatable gantry 2 and are symmetrically disposed with the treatment heads 3, 3′, for keeping the rotatable gantry balance during revolution.

The radiotherapeutic apparatus according to the present invention improves the focal skin dose ratio, by enabling two treatment heads 3, 3′ to swing along the axial direction of the rotatable gantry and to move on two spatial revolution surfaces. In addition, a combination of the two treatment heads 3, 3′ may also improve the dose ratio at a tumor target.

Described above are merely preferred embodiments of the present invention, but are not intended to limit the present invention. Any modification, equivalent replacement, or improvement made without departing from the spirit and principle of the present invention should fall within the protection scope of the present invention.

What is claimed is:

1. A radiotherapeutic apparatus, comprising a base, a rotatable gantry, two treatment heads and a treatment table, the rotatable gantry being disposed on the base, and the treatment heads being disposed on the rotatable gantry, wherein the radiotherapeutic apparatus further comprises:
two groups of driving units, disposed on the rotatable gantry and coupled to the two treatment heads respectively;
two groups of gears, disposed on the rotatable gantry and coupled to the driving units respectively, and the driving units driving the gears to rotate; and
two groups of arc-shaped racks, disposed on the treatment heads and meshing with the two groups of gears respectively;
wherein, when the gears are driven under driving of the driving units, each treatment head which is locked to the arc-shaped racks separately slide on the arc-shaped racks, along an axial direction of the rotatable gantry.

2. The radiotherapeutic apparatus according to claim 1, further comprising:
arc-shaped guide rails, disposed on the rotatable gantry, a center of circle of the arc-shaped guide rail coinciding with a center of circle of the arc-shaped rack; and
at least one sliding block, disposed on the treatment heads, the sliding block being slidable on the arc-shaped guide rail.

3. The radiotherapeutic apparatus according to claim 1, wherein an angle of the swinging along the axial direction of the rotatable gantry is in a range from 0 to 40 degrees.

4. The radiotherapeutic apparatus according to claim 1, wherein the treatment head comprises a radioactive source.

5. The radiotherapeutic apparatus according to claim 4, wherein the radioactive source comprises a cobalt radioactive source.
